(19) United States
(12) Patent Application Publication
(10) Pub. No.: US 2011/0229420 A1
(43) Pub. Date: Sep. 22, 2011

(54) Cleaning Composition Containing Substituted Starch

(76) Inventors: Hiroshi OH, (US); Lee Arnold Schechtman, Fairfield, OH (US); Joseph Jay Kemper, Cincinnati, OH (US); Ming Tang, Beijing (CN); Kenneth Nathan Price, Cincinnati, OH (US); Yonas Gizaw, Cincinnati, OH (US); Steven Daryl Smith, Fairfield, OH (US)

(21) Appl. No.: 13/049,253

(22) Filed: Mar. 16, 2011

Related U.S. Application Data
(63) Continuation of application No. PCT/US2009/057651, filed on Sep. 21, 2009.
(60) Provisional application No. 61/098,308, filed on Sep. 19, 2008.

(51) Int. Cl. A61K 8/73; C08B 31/00; C11D 3/37; C11D 17/00; A61Q 11/00; A61Q 5/02

(52) U.S. Cl. 424/49; 536/102; 510/276; 510/218; 510/474; 510/404; 510/119

(57) ABSTRACT

A cleaning composition having from 0.05% to 30% by weight of surfactant and from 0.01% to 10% by weight of substituted starch, wherein the substituted starch has:
a total degree of substitution in the range of from 0.001 to 0.6,
a degree of substitution of anionic substituent in the range of from 0.001 to 0.04 and/or a degree of substation of nonionic substituent in the range of from 0.01 to 0.5,
a degree of substitution of cationic substituent in the range of from 0 to 0.004,
an average per weight molecular weight in the range of from 10 000 to 100 000 000 Daltons.

### CROSS REFERENCE TO RELATED APPLICATION(S)

This application is a Continuation of International Application No. PCT/US2009/057651, filed Sep. 21, 2009, which claims the benefit of U.S. Provisional Application Ser. No. 61/098,308, filed Sep. 19, 2008.

### FIELD OF THE INVENTION

The present invention relates to a cleaning composition comprising starch substituted with anionic and/or nonionic substituent. More particularly, the substituted starch in the present invention provide suds boosting benefit, such as suds volume and suds duration enhancement, to cleaning compositions such as fabric care products, dish care products, oral care product, or applications where cleaning of surfaces is needed.

### BACKGROUND OF THE INVENTION

Cleaning compositions must satisfy several criteria in order to be effective and fulfill the need of the consumer. In particular, the presence of suds in a cleaning operation has long been used as a signal that the detergent continues to be effective. However, depending upon the circumstances, the presence of suds or the lack thereof, may have little or no bearing upon the efficacy of the cleaning process. Therefore, the consumer who relies upon a somewhat erroneous signal may tend to use an excess of cleaning product in the lack or absence of suds.

Accordingly, there remains a need for adjusting the sudsing properties of a cleaning composition with flexibility, in particular to adjust the sudsing properties relatively independently from the cleaning properties. This is especially needed for cleaning composition comprising a low level of surfactant or of builder.

The inventors have discovered that some or all of the above mentioned needs could be at least partially fulfilled in the cleaning composition of the invention by using a specific substituted starch.

Unless otherwise specified, all percentage and ratio are in weight

### SUMMARY OF THE INVENTION

The present disclosure relates to a cleaning composition comprising from 0.05% to 30% by weight of surfactant and from 0.01% to 10% by weight of substituted starch polymer, wherein the substituted starch has:
a total degree of substitution in the range of from 0.001 and 0.6,
a degree of substitution of anionic substituent in the range of from 0.001 to 0.04 and/or a degree of substation of nonionic substituent in the range of from 0.01 to 0.5,
a degree of substitution of cationic substituent in the range of from 0 to 0.004,
an average per weight molecular weight in the range of from 10 000 to 100 000 000 Daltons.

According to an embodiment of the invention, the invention concerns the use of a substituted starch, wherein the substituted starch has:
a total degree of substitution in the range of from 0.001 and 0.6,
a degree of substitution of anionic substituent in the range of from 0.001 and 0.04 and/or a degree of substation of nonionic substituent in the range of from 0.01 and 0.5,
a degree of substitution of cationic substituent in the range of from 0 to 0.004,
an average per weight molecular weight in the range of from 10 000 to 100 000 000 Daltons,

to increase the quantity of suds that can be generated by a cleaning composition.

### DETAILED DESCRIPTION OF THE INVENTION

### Starch

The cleaning composition of the invention comprises from 0.01% to 10% by weight of one or more substituted starch. Preferably, the cleaning composition of the invention comprises from 0.1% to 7% by weight of substituted starch, in particular from 0.1% to 5%, typically from 0.3% to 3% by weight of substituted starch polymer.

The starch polymer comprises sugar monomers. The sugar monomers can be substituted or not substituted. When the sugar monomer is substituted, one or more of its hydroxy function may be substituted.

The substituted starch of the invention has a total degree of substitution (DSt) in the range of from 0.001 to 0.6. Typically, the substituted starch has a DSt of in the range of from 0.003 to 0.3, in particular in the range of from 0.01 to 0.2.

DSt corresponds to the ratio between the sugar monomers which are substituted to the total number of sugar monomers of the starch polymer. If all the sugar monomers are substituted at least once, the value of DSt is 1.

The substituted starch of the invention has a DSt in the range of from 0.001 and 0.6, typically between 0.01 and 0.5.

For the purpose of this disclosure, degree of substitution of a specific substituent refers to the ratio of sugar momomers which are substituted at least once with said specific substituent to the total number of sugar monomers of the starch polymer.

DSa corresponds to the ratio between the sugar monomers of the starch polymer which are substituted with at least one anionic substituent and the total number of sugar monomers of the starch polymer. If all the sugar monomers are substituted with at least one anionic substituent, the value of DSa is 1.

DSn corresponds to the ratio between the sugar monomers of the starch polymer which are substituted with at least one nonionic substituent and the total number of sugar monomers of the starch polymer. If all the sugar monomers are substituted with at least one nonionic substituent, the value of DSn is 1.

DSc corresponds to the ratio between the sugar monomers of the starch polymer which are substituted with at least one cationic substituent and the total number of sugar monomers of the starch polymer. If all the sugar monomers are substituted with at least one cationic substituent, the value of DSc is 1.

For the purpose of this disclosure, a substituent is considered anionic, cationic or nonionic, depending of its net charge value at a pH of 10.

The substituted starch of the invention has a degree of substitution of anionic substituent (DSa) in the range of from 0.001 to 0.04 and/or a degree of substitution of nonionic substituent (DSn) in the range of from 0.01 to 0.5. The substituted starch of the invention may have a degree of substitution of anionic substituent in the range of from 0.001 to 0.04, preferably in the range of from 0.003 to 0.02, in particular in the range of from 0.01 to 0.01. The substituted starch of the invention may have a degree of substation of nonionic substituent in the range of from 0.01 and 0.5, preferably in the range of from 0.03 to 0.3, in particular in the range of from 0.08 to 0.15.

The substituted starch of the invention has a degree of substitution of cationic substituent DSc in the range of from 0 to 0.004. Preferably, the substituted starch has a DSc in the range of from 0 to 0.02, typically in the range of from 0 to 0.001 or from 0 to 0.0005 or from 0 to 0.0002 or even from 0 to 0.0001 or from 0 to 0.00002. Preferably, the DSc is below the DSa. As such, the substituted starch has preferably an overall net negative charge. Typically, the ratio DSc to DSa, per number of substituent, is below 1, typically below 0.9 or 0.75 or 0.5, or even below 0.3 or 0.1 or 0.05 or 0.01 or 0.001.

The inventors have found that substituted starch comprising a low DSc, in particular having a DSc below the DSa, were particularly suitable for providing the sudsing benefit of the invention.

The substituted starch of the invention has an average per weight molecular weight in the range of from 10 000 to 100 000 000 Daltons. Preferably the substituted starch has an average per weight molecular weight of from 20 000 to 50 000 000 Daltons.

Preferably, when the substituted starch has a DSa above 0.001, for example above 0.005 or 0.01, the substituted starch has an average per weight molecular weight in the range of from 20 000 to 500 000. Daltons, preferably in the range of from 40 000 to 200 000 or from 70 000 to 150 000.

According to an exemplary embodiment of the invention, the substituted starch has a DSa above 0.01, and a DSc above 0.01. Preferably, when the substituted starch has a DSa above 0.01, and a DSc above 0.01, the substituted starch has an average per weight molecular weight in the range of from 100 000 to 100 000 000 Daltons, preferably in the range of from 200 000 to 50 000 000 or from 500 000 to 10 000 000 or even from 1 000 000 to 5 000 000.

The substituted starch may have a degree of substitution of nonionic hydroxyalkyl substituent in the range of from 0.01 and 0.5. In particular, the substituted starch has a degree of substitution of nonionic hydroxyalkyl substituent in the range of from 0.03 to 0.3, or from 0.08 to 0.15.

The substituted starch may have a degree of substitution of anionic substituent comprising a sulphate or sulphonate group in the range of from 0.001 and 0.4, preferably in the range of from 0.003 to 0.1, in particular in the range of from 0.005 to 0.2.

The inventors have found that starches substituted with anionic substituent comprising a sulphate or sulphonate group or with nonionic hydroxyalkyl substituent were particularly suitable to provide the sudsing benefit of the invention.

Starch comprises amylose (see formula I below) and amylopectin (see formula II below). Starch is described in Kirk-Othmer's Encyclopedia of Chemical Technology 4^{th} Edition, Vol. 22, at pp. 699-719.

The substituted starch may comprise substituted polymers selected from polymers of general formula:

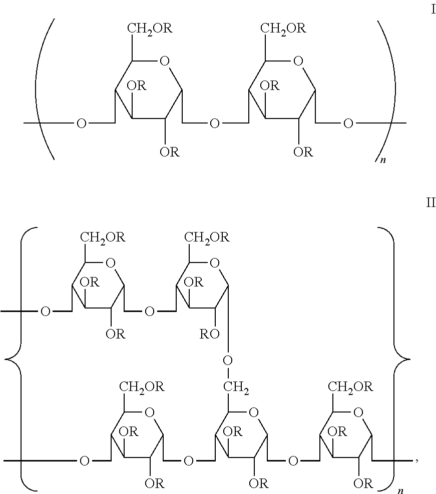

and mixtures thereof, wherein each R is H or a substituent selected from the group of anionic substituent, nonionic substituent, and cationic substituent, wherein at least 70% by number of the R are H, the degree of substitution of anionic substituent is in the range of from 0 to 0.04, the degree of substitution of nonionic substituent is in the range of from 0 to 0.5, the degree of substitution of cationic substituent is in the range of from 0 to 0.004, wherein either the degree of substitution of anionic substituent is of at least 0.002 or the degree of substitution of nonionic is of at least 0.02, wherein n is such that said suds boosting substituted polymer(s) has an average per weight molecular weight ranging from 20 000 Daltons to 100 000 000 Daltons.

Suitable anionic, nonionic, or cationic substituents may be selected from the group consisting of branched, linear or cyclic, substituted or not substituted, saturated or unsaturated alkyl, amine (primary, secondary, tertiary), ammonium salt, amide, urethane, alcohol, carboxylic acid, tosylate, sulfonate, sulfate, nitrate, phosphate, silicone, and mixtures thereof.

The anionic, nonionic, and cationic substituents may be selected from the group consisting of: R₁, N(R₂)(R₃), silicone moiety, SO₃⁻, PO₃⁻, with R₂ and R₃ being independently of each other an hydrogen atom or a C₁₋₆ alkyl and R₁ being a linear or branched, typically linear, saturated or unsaturated, typically saturated, substituted or unsubstituted, typically substituted, cyclic or acyclic, typically acyclic, aliphatic or aromatic, typically aliphatic, C₁-C₃₀₀, typically C₁-C₃₀, C₁-C₁₂, or C₁-C₆ hydrocarbon radical which hydrocarbon backbone may be interrupted by a heteroatom chosen form O, S, N and P. R₁ may be substituted by one or more radical selected from amino (primary, secondary, or tertiary), amido, —OH, —CO—OR₄, —SO₃⁻, R₄, —CN, and —CO—R₄, where R₄ represents a hydrogen atom or an alkali metal, preferably a sodium or potassium, ion.

The anionic substituent may be one following anionic groups, in its acid or salt form, preferably sodium (given here) or potassium salt form:

-T-CO₂Na

-T-SO₃Na

—PO₃Na

—SO₃Na

Wherein T is a C₁₋₆ alkyl, more preferably C₁₋₄ alkyl.

The cationic substituent may be the following cationic group:

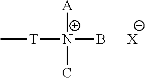

Wherein T is a C₁₋₆ alkyl, or CH₂CH(OH)CH₂, each A, B, and C is C₁₋₆ alkyl or hydroxy-C₁₋₆ alkyl, X is a counterion such as halide or tosylate.

The nonionic substituent may be one following non-ionic groups:

-A

-T-OH

-T-CN

—C(═O)A

—C(═O)NH₂

—C(═O)NHA

—C(═O)N(A)B

—C(═O)OA

—(CH₂CH₂CH₂O)ₙZ

—(CH₂CH₂O)ₙZ

—(CH₂CH(CH₃)O)ₙZ

—(CH₂O)ₙZ

Wherein: A and B are C₁₋₃₀ alkyl; T is C₁₋₆ alkyl; n=1 to 100; Z is H or C₁₋₆ alkyl.

Preferably the anionic substituent comprises a sulphate or sulphonate group. Preferably the nonionic substituent is a nonionic hydroxyalkyl wherein the alkyl is a C₁-C₆ alkyl, in particular a C₁-C₄ or C₁-C₃ or C₁-C₂ alkyl.

Preferably, the ratio of the degree of substitution of anionic substituent comprising a sulphate or sulphonate group to the total degree of substitution of anionic substituent is in the range of from 1:5 to 1:1, preferably above 1:3 or 1:2 or even 1:1.5 or 1:1.2 or 1:1.1.

Preferably, the ratio of the degree of substitution of nonionic hydroxyalkyl substituent wherein the alkyl is a C₁-C₆ alkyl, to the total degree of substitution of nonionic substituent is in the range of from 1:5 to 1:1, preferably above 1:3 or 1:2 or even 1:1.5 or 1:1.2 or 1:1.1.

Surfactant

The cleaning composition of the invention comprises from 0.05% to 30% by weight of one or more surfactant(s). Preferably, the cleaning composition comprises from 0.1% to 25% of surfactant, typically from 0.5% to 20%, or from 1% to 15% by weight of surfactant. The surfactant may be anionic, nonionic and/or cationic. The substituted starch of the invention is particularly efficient in the presence of anionic surfactant.

The weight ratio of substituted starch to surfactant may be in the range of from 1:100 to 1:3, in particular from 1:50 to 1:5, or from 1:40 to 1:10, or from 1:30 to 1:20.

The weight ratio of substituted starch to anionic surfactant may be in the range of from 1:100 to 1:3, in particular from 1:50 to 1:5, or from 1:40 to 1:10, or from 1:30 to 1:20.

The weight ratio of nonionic surfactant to anionic surfactant may be in the range of from 1:100 to 1:1, in particular from 1:50 to 1:5, for example below 1:10 or 1:20.

Anionic Surfactant

The cleaning composition of the invention may comprise from 0.05% to 30% by weight of one or more anionic surfactant. The cleaning composition may comprise 0.1% to 25%, in particular from 0.5% to 20% or from 1% to 18%, or even from 1.5% to 16% or from 2% to 14% by weight of anionic surfactant.

In particular, the anionic surfactant may comprise anionic surfactants selected from alkyl ester sulfonate(s); linear, branched, and modified alkylbenzene sulfonate(s); C₁₀-C₁₈ alkyl alkoxy sulfates; C₁₀₋₂₀ primary, branched-chain and random alkyl sulfates; C₁₀-C₁₈ secondary (2,3) alkyl sulfates; C₁₀-C₁₈ alkyl alkoxy carboxylate(s); fatty acid(s); mid-chain branched alkyl sulfate(s); mid-chain branched alkyl alkoxy sulfate(s); alpha-olefin sulfonate(s); phosphate ester(s); and mixtures thereof.

The anionic surfactant may comprise surfactant chosen among sarcosinate surfactants, isethionate surfactants and taurate surfactants. Preferred for use herein are alkali metal or ammonium salts of these surfactants. Most preferred herein are the sodium and potassium salts of the following: lauroyl sarcosinate, myristoyl sarcosinate, palmitoyl sarcosinate, stearoyl sarcosinate and oleoyl sarcosinate.

Non-Ionic Surfactant

The cleaning composition may comprise non-ionic surfactant. Where present the non-ionic detersive surfactant(s) is generally present in amounts of from 0.01 wt % to 20 wt %, or from 0.1 wt % to 4 wt % by weight of the cleaning composition.

The non-ionic detersive surfactant can be selected from the group consisting of: alkyl polyglucoside and/or an alkyl alkoxylated alcohol; C₁₂-C₁₈ alkyl ethoxylates, such as, NEODOL® non-ionic surfactants from Shell; C₆-C₁₂ alkyl phenol alkoxylates wherein the alkoxylate units are ethyleneoxy units, propyleneoxy units or a mixture thereof; C₁₂-C₁₈ alcohol and C₆-C₁₂ alkyl phenol condensates with ethylene oxide/propylene oxide block polymers such as Pluronic® from BASF; C₁₄-C₂₂ mid-chain branched alcohols, BA, as described in more detail in U.S. Pat. No. 6,150,322; C₁₄-C₂₂ mid-chain branched alkyl alkoxylates, BAEx, wherein x=from 1 to 30, as described in more detail in U.S. Pat. No. 6,153,577, U.S. Pat. No. 6,020,303 and U.S. Pat. No. 6,093,856; alkylpolysaccharides as described in more detail in U.S. Pat. No. 4,565,647, specifically alkylpolyglycosides as described in more detail in U.S. Pat. No. 4,483,780 and U.S. Pat. No. 4,483,779; polyhydroxy fatty acid amides as described in more detail in U.S. Pat. No. 5,332,528, WO 92/06162, WO 93/19146, WO 93/19038, and WO 94/09099; ether capped poly(oxyalkylated) alcohol surfactants as described in more detail in U.S. Pat. No. 6,482,994 and WO 01/42408; and mixtures thereof.

Cationic Detersive Surfactant

The cleaning composition may comprise a cationic detersive surfactant. When present, preferably the cleaning composition comprises from 0.01 wt % to 10 wt %, or from 0.1 wt % to 2 wt % cationic detersive surfactant.

Suitable cationic detersive surfactants are alkyl pyridinium compounds, alkyl quaternary ammonium compounds, alkyl quaternary phosphonium compounds, and alkyl ternary sulphonium compounds. The cationic detersive surfactant can be selected from the group consisting of: alkoxylate quaternary ammonium (AQA) surfactants as described in more detail in U.S. Pat. No. 6,136,769; dimethyl hydroxyethyl quaternary ammonium surfactants as described in more detail in U.S. Pat. No. 6,004,922; polyamine cationic surfactants as described in more detail in WO 98/35002, WO 98/35003, WO 98/35004, WO 98/35005, and WO 98/35006; cationic ester surfactants as described in more detail in U.S. Pat. No. 4,228,042, U.S. Pat. No. 4,239,660, U.S. Pat. No. 4,260,529 and U.S. Pat. No. 6,022,844; amino surfactants as described in more detail in U.S. Pat. No. 6,221,825 and WO 00/47708, specifically amido propyldimethyl amine; and mixtures thereof.

cationic detersive surfactants may be chosen among mono-C₈₋₁₀ alkyl mono-hydroxyethyl di-methyl quaternary ammonium chloride, mono-C₁₀₋₁₂ alkyl mono-hydroxyethyl di-methyl quaternary ammonium chloride and mono-C₁₀ alkyl mono-hydroxyethyl di-methyl quaternary ammonium chloride. Cationic surfactants such as Praepagen HY (tradename Clariant) may be useful and may also be useful as a suds booster.

Builder

The cleaning composition of the invention may comprise a builder. When a builder is used, the cleaning composition will typically comprise from 1% to about 40%, typically from 2 to 20%, or even from about 4% to about 15%, or from 5 to 10% by weight of builder(s).

The composition may further comprise from 1% to about 40%, typically from 2 to 20%, or even from about 4% to about 15%, or from 5 to 10% by weight of builder(s), chelant(s), or, in general, any material which will remove calcium ions from solution by, for example, sequestration, complexation, precipitation or ion exchange.

The composition may comprise a chelant. Suitable chelants include diethylene triamine pentaacetate, diethylene triamine penta(methyl phosphonic acid), ethylene diamine-N′N′-disuccinic acid, ethylene diamine tetraacetate, ethylene diamine tetra(methylene phosphonic acid) and hydroxyethane di(methylene phosphonic acid). A preferred chelant is ethylene diamine-N′N′-disuccinic acid (EDDS) and/or hydroxyethane diphosphonic acid (HEDP). Preferably the ethylene diamine-N′N′-disuccinic acid is in S′S′ enantiomeric form. The composition of the invention may comprise less than 3% or less than 2% or 1% or 0.5% of each of the above mentioned chelants.

Builders include, but are not limited to, the alkali metal, ammonium and alkanolammonium salts of polyphosphates, alkali metal silicates, layered silicates, such as SKS-6 of Clariant®, alkaline earth and alkali metal carbonates, aluminosilicate builders, such as zeolite, and polycarboxylate compounds, ether hydroxypolycarboxylates, copolymers of maleic anhydride with ethylene or vinyl methyl ether, 1,3,5-trihydroxy benzene-2,4,6-trisulphonic acid, and carboxymethyloxysuccinic acid, fatty acids, the various alkali metal, ammonium and substituted ammonium salts of polyacetic acids such as ethylenediamine tetraacetic acid and nitrilotriacetic acid, as well as polycarboxylates such as mellitic acid, succinic acid, citric acid, oxydisuccinic acid, polymaleic acid, benzene 1,3,5-tricarboxylic acid, carboxymethyloxysuccinic acid, and soluble salts thereof.

The cleaning composition may comprise less than 50%, in particular less than 25% or less than 20%, 15%, 10%, or 5% by weight of phosphate and/or aluminosilicate builders.

The cleaning composition may comprise from 0 to 50%, in particular from 1% to 25%, or less than 20%, or less than 15%, or less than 10%, or less than 5%, or less than 1% by weight of phosphate builder(s).

The cleaning composition may comprise from 0 to 50%, in particular from 1% to 25%, or less than 20%, or less than 15%, or less than 10%, or less than 5%, or less than 1% by weight of aluminosilicate builder(s). The aluminosilicate builder may comprise zeolite.

The cleaning composition may comprise from 0 to 50%, in particular from 1% to 25%, or less than 20%, or less than 15%, or less than 10%, or less than 5%, or less than 1% by weight of polycarboxylic acid(s) and salt(s) thereof.

The cleaning composition may comprise from 0 to 50%, in particular from 1% to 25%, or less than 20%, or less than 15%, or less than 10%, or less than 5%, or less than 1% by weight of layered silicate(s).

The cleaning compositions of the present invention may comprise from 0 to 50%, in particular from 1% to 25%, or less than 20%, or less than 15%, or less than 10%, or less than 5%, or less than 1% by weight of sodium carbonate.

### Adjunct Ingredient

The cleaning composition may comprise one or more adjunct ingredient(s). The precise nature of these additional adjunct components, and levels of incorporation thereof, will depend on the physical form of the composition and the nature of the operation for which it is to be used.

For example when the composition is a fabric care composition, suitable adjunct materials include, but are not limited to flocculating aid, chelating agents, dye transfer inhibitors, enzymes, enzyme stabilizers, catalytic materials, bleach activators, hydrogen peroxide, sources of hydrogen peroxide, preformed peracids, polymeric dispersing agents, clay soil removal/anti-redeposition agents, brighteners, suds suppressors, dyes, perfumes, structure elasticizing agents, fabric softeners, carriers, hydrotropes, processing aids, and/or pigments. In addition to the disclosure below, suitable examples of such other adjuncts and levels of use are found in U.S. Pat. Nos. 5,576,282, 6,306,812 B1 and 6,326,348 B1.

The cleaning composition may also comprise, in particular when the cleaning composition is an oral care composition, Anticalculus Agent, Fluoride Source, Thickening Agents, Flavoring and Sweetening Agents, and/or antimicrobial agents.

Preferably, the cleaning composition of the invention contains less than 3%, preferably up to 1%, and most preferably less than 0.1% or less than 0.01% or 0.001% or even 0.0001% of suds suppressor selected from the group consisting of trimethyl-, diethyl-, dipropyl-, dibutyl-, methylethyl-, phenylmethyl polysiloxane, and mixtures thereof. Preferably, the compositions of the invention contain less than 3%, preferably up to 1%, and most preferably less than 0.1% or less than 0.01% or 0.001% or even 0.0001% of suds suppressor.

### Cleaning Composition

The cleaning composition may be in any liquid or solid form, in the form of gel, paste, dispersion, preferably a colloidal dispersion or any combination thereof. The cleaning composition is preferably in a solid form or in the form of a paste. The cleaning composition may be in particulate form, for example in free-flowing particulate form. The composition in solid form can be in the form of an agglomerate, granule, flake, extrudate, bar, tablet or any combination thereof.

The cleaning composition may be capable of cleaning and/or softening fabric during a laundering process. The cleaning composition may be an oral care composition. The Oral care composition may be in the form of a toothpaste, dentifrice, tooth powder, tooth gel, subgingival gel, mouthrinse, denture product, mouthspray, lozenge, oral tablet, or chewing gum. The oral composition may also be incorporated onto strips or films for direct application or attachment to oral surfaces. The cleaning composition may be a hair care, or a dish care composition.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

The following examples are given by way of illustration only and therefore should not be construed to limit the scope of the invention.

### EXAMPLES

### Example 1

### Oral Care Composition

| Ingredients | Function | 1A | 1B | 1C | 1D |
|---|---|---|---|---|---|
| Silica, dental type | Abrasive (cleaning | 15.0 | 15.0 | 15.0 | 15.0 |
| | agent) | | | | |
| NaF USP | Fluoride source for | 0.243 | 0.243 | 0.243 | 0.243 |
| | anti-caries benefit | | | | |
| Sodium dodecyl phosphate | Anionic surfactant with | — | 5.0 | 5.0 | 10.0 |
| (30% soln) | functional properties | | | | |
| Zinc citrate | Antimicrobial | 0.5 | — | — | — |
| Sodium acid pyrophosphate | Antitartar agent | 4.17 | — | — | — |
| Sodium saccharin | Sweetener | 0.13 | 0.13 | 0.13 | 0.13 |
| NaOH (50% soln) | pH adjuster | 0.5 | 0.5 | 0.5 | 1.0 |
| CMC sodium | Thickener | 0.75 | 0.75 | 0.75 | 0.75 |
| Titanium dioxide | Opacifier | 0.25 | 0.25 | 0.25 | 0.25 |
| Carbomer 956 | Thickener | 0.30 | 0.30 | 0.30 | 0.30 |
| Flavor | Flavor | 0.80 | 0.80 | 0.80 | 0.80 |
| Sodium lauryl sulfate | Main surfactant for | | | | |
| (28% soln) | Foaming | 4.0 | 4.0 | 3.3 | — |
| Cocamidopropyl Betaine | Co-surfactant for | — | — | | — |
| (30% soln.) | foaming | | | | |
| Sorbitol solution | Humectant and Carrier | 60 | 60 | 60 | 60 |
| | (vehicle) | | | | |
| Hydroxy Butyl Starch | Foam Stabilizer | 0.80 | | 0.70 | |
| (MW 2,812 kDa, DS 0.066)) | | | | | |
| Sulfonated Starch (MW 47 | Foam Stabilizer | | | | 0.90 |
| kDa, DS 0.001) | | | | | |
| Carboxy Methyl Starch with | Foam Stabilizer | | 0.60 | | |
| low DS of Quat (MW 80,000 | | | | | |
| kDa, DS 0.44) | | | | | |
| FD&C Blue #1 | Visual | 0.05 | 0.05 | 0.05 | 0.05 |
| USP Water | Carrier (vehicle) | Q.S | Q.S | Q.S | Q.S |

### Example 2

### Fabric Care Compositions

| | Example 2A | Example 2B | Example 2C | Example 2D |
|---|---|---|---|---|
| Component | Concentration (Weight percents) | | | |
| Sodium LAS | 12 | 14 | 8 | 5 |
| Sodium AE3S | 2 | | | |
| Sodium AE1S | | 2 | | 3 |
| Hydroxy Butyl | 0.80 | | 0.70 | |
| Starch (MW 2,812 | | | | |
| kDa, DS 0.066)) | | | | |
| Sulfonated Starch | | | | 0.90 |
| (MW 47 kDa, | | | | |
| DS 0.001) | | | | |
| Carboxy Methyl | | 0.60 | | |
| Starch with low | | | | |
| DS of Quat | | | | |
| (MW 80,000 | | | | |
| kDa, DS 0.44) | | | | |
| Cationic | 0.7 | 0.8 | 0.6 | 0.5 |
| surfactant | | | | |
| Non ionic | 0.3 | 0.3 | | |
| surfactant | | | | |
| Zeolite | 4 | 3 | | |
| Sodium carbonate | 32 | 25 | 35 | 34 |
| Silicate | 3 | 4 | 7 | 8 |
| polymers | 12 | 11 | 15 | 14 |
| enzyme | 2 | 1.5 | 2.5 | 2.6 |
| Bleach system | 6 | 5 | 8 | 10 |
| Sodium sulfate | 15 | 24 | 12 | 15 |
| Water and | bal | bal | bal | bal |
| miscelaneous | | | | |

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A cleaning composition comprising from 0.05% to 30% by weight of surfactant and from 0.01% to 10% by weight of substituted starch, wherein the substituted starch has:
a total degree of substitution in the range of from 0.001 to 0.6,
a degree of substitution of anionic substituent in the range of from 0.001 to 0.04 and/or a degree of substation of nonionic substituent in the range of from 0.01 to 0.5,
a degree of substitution of cationic substituent in the range of from 0 to 0.004,
an average per weight molecular weight in the range of from 10 000 to 100 000 000 Daltons.

2. A cleaning composition according to claim 1, comprising from 0.1% to 25% by weight of surfactant.

3. A cleaning composition according to claim 1, comprising from 0.1% to 7% by weight of surfactant of substituted starch.

4. A cleaning composition according to claim 1, wherein the weight ratio of substituted starch to surfactant is in the range of from 1:100 to 1:3.

5. A cleaning composition according to claim 1, comprising less than 50% by weight of phosphate and/or aluminosilicate builders.

6. A cleaning composition according to claim 1, wherein the substituted starch comprises substituted polymers selected from polymers of general formula: 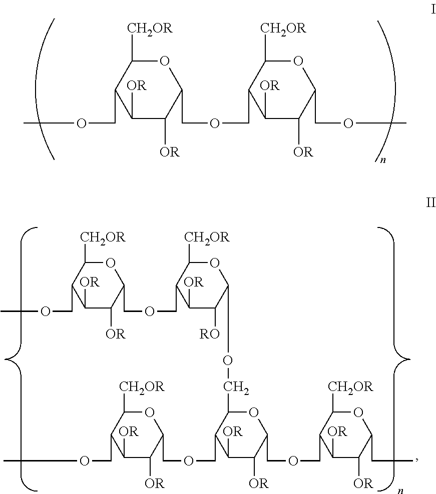
and mixtures thereof, wherein each R is H or a substituent selected from the group of anionic substituent, nonionic substituent, and cationic substituent, wherein at least 70% by number of the R are H, the degree of substitution of anionic substituent is in the range of from 0 to 0.04, the degree of substitution of nonionic substituent is in the range of from 0 to 0.5, the degree of substitution of cationic substituent is in the range of from 0 to 0.004, wherein either the degree of substitution of anionic substituent is of at least 0.002 or the degree of substitution of non-ionic is of at least 0.02, wherein n is such that said suds boosting substituted polymer(s) has an average per weight molecular weight ranging from 20 000 Daltons to 100 000 000 Daltons.

7. A cleaning composition according to claim 1, wherein the substituted starch has:
a degree of substitution of anionic substituent in the range of from 0.001 and 0.04,
a degree of substitution of cationic substituent below the degree of substitution of anionic substituent,
an average per weight molecular weight in the range of from 20 000 to 500 000 Daltons.

8. A cleaning composition according to claim 7, wherein the substituted starch has a degree of substitution of anionic substituent comprising a sulphate or sulphonate group in the range of from 0.001 and 0.04.

9. A cleaning composition according to claim 1, wherein the substituted starch has:
a degree of substitution of nonionic substituent in the range of from 0.01 and 0.5,
a degree of substitution of cationic substituent in the range of from 0 to 0.0009,
an average per weight molecular weight in the range of from 20 000 and 50 000 000 Daltons.

10. A cleaning composition according to claim 9, wherein the substituted starch has a degree of substitution of nonionic hydroxyalkyl substituent in the range of from 0.01 and 0.5.

11. A cleaning composition according to claim 1, comprising less than 3%, in particular less than 1% or less than 0.1% of suds suppressor.

12. A cleaning composition according to claim 1, being a fabric care product selected from the group consisting of liquid laundry detergents, solid laundry detergents, laundry soap products, laundry spray treatment products, or a dish washing detergent, a beauty care detergent, a shampoo, an oral care composition, or a household cleaning detergent.

13. A cleaning composition according to claim 1 being in the form of a paste or a solid.

14. A cleaning composition according to claim 1 being a fabric care or an oral care composition.

15. Use of a substituted starch, wherein the substituted starch has:
a total degree of substitution in the range of from 0.001 and 0.6,
a degree of substitution of anionic substituent in the range of from 0.001 and 0.04 and/or a degree of substation of nonionic substituent in the range of from 0.01 and 0.5,
a degree of substitution of cationic substituent in the range of from 0 to 0.004,
an average per weight molecular weight in the range of from 10 000 to 100 000 000 Daltons,
to increase the quantity of suds that can be generated by a cleaning composition.
